(19) United States
(12) Patent Application Publication
(10) Pub. No.: US 2011/0237973 A1
(43) Pub. Date: Sep. 29, 2011

(54) METHOD FOR USING A SILVER-SILVER CHLORIDE NEEDLE ELECTRODE SYSTEM

(76) Inventor: DAVID A. SHERATON, SR., Aliso Viejo, CA (US)

(21) Appl. No.: 13/158,306

(22) Filed: Jun. 10, 2011

Related U.S. Application Data
(62) Division of application No. 12/401,769, filed on Mar. 11, 2009.

(51) Int. Cl. A61B 5/053

(52) U.S. Cl. 600/547

(57) ABSTRACT

A method for using a needle electrode system for acquiring biopotential signals from a person's body. The needle electrode system comprises at least one silver-silver chloride needle electrode and at least one connector coupled with a monitoring device. The needle electrode system senses and sends biopotential signals to the monitoring device in preference to cutaneous electrode on the person's body by inserting the needle electrode subcutaneously into the person's body.

This is a divisional application of the co-pending application Ser. No. 12/401,769.

### BACKGROUND OF THE INVENTION

1. TECHNICAL FIELD OF THE INVENTION

The present invention relates in general to a method for using a needle electrode system having a needle electrode made out of or coated with silver-silver chloride. More specifically, the present invention relates to a method for using a needle electrode system for acquiring biopotential signals from a person's body.

2. DESCRIPTION OF THE RELATED ART

The advancement of needle electrodes has been revolutionizing the modern medical diagnosis and treatment over a long time. Needle electrodes are invasive bio-electrodes that are inserted into a person's body for acquiring biopotential signals. Needle electrodes sense and collect electrophysiological information such as ECG, EEG, EMG and intracellular potentials. These devices transform biochemical phenomena into electrical currents. The presence, shape, and size of a waveform produced on an electronic monitoring device provide information about the ability of the person's body to respond to a nervous stimulation.

Several biopotential monitoring systems utilize surface electrodes for sensing and sending biopotential signals. Surface electrodes are placed on a person's body for gathering biopotential signals. A common problem associated with surface electrodes is the susceptibility of interference to the acquired signal from adjacent electrodes. Moreover, the signals acquired through surface electrodes are prone to crosstalk.

One type of needle electrode system that can be inserted into a person's body for sensing biopotential signals overcomes the drawbacks of the surface electrodes. These needle electrodes are made with stainless steel. However, these needle electrodes are polarizable and are susceptible to charge build up, when monitoring low frequency signals. As a result, the impedance remains high thereby rendering the system incapable to sense and send accurate biopotential signals to a monitoring device.

A type of needle electrode system for recording facial EMG utilizes a pair of needle electrodes. Such a mechanism causes inconvenience to a patient while monitoring EMG signals. Furthermore, such a mechanism requires detachable parts that are complex to manufacture.

Another needle electrode assembly includes a holder that releasably holds needle electrodes of various sizes, shapes, and lengths. The needle electrode-holder is operated by placing the needle electrode holder in a hand and depressing the base of the holder to release or insert a needle electrode. The needle electrode is inserted into the tip of the needle electrode holder while depressing the base of the needle electrode holder. However, the assembly requires an internal engagement mechanism of the needle electrode holder and a gripping assembly located within the body to engage and hold the needle in place. In addition, the needle electrode holder is to be held in a person's hand in a recommended position causing a relatively complex procedure to acquire the biopotential signals from a person's body.

Many attempts are made to manufacture simple needle assembly with lesser detachable parts. Such an attempt introduced a needle assembly with a non detachable holder. This approach is difficult and expensive to manufacture.

Hence, it can be seen that there is a need for a needle electrode system that would be capable of sensing and sending biopotential signals from very small muscles without interference and cross talk. Such a system would be non-polarizable and capable of dissipating charge built up when monitoring low frequency signals. Further, the system would reduce the impedance offered by the person's body thereby senses and sends accurate biopotential signals.

### SUMMARY OF THE INVENTION

To minimize the limitations found in the prior art, and to minimize other limitations that will be apparent upon the reading of the specifications, the present invention provides a method for using a needle electrode system for acquiring biopotential signals from a person's body. The needle electrode system comprises at least one silver-silver chloride needle electrode that is coupled to a distal end of a first wire, at least one cutaneous electrode connector coupled to a distal end of a second wire and at least one connector attached to at least one proximal end of the first wire and the second wire. The connector is coupled with a monitoring device. The needle electrode system senses and sends biopotential signals to the monitoring device in preference to the cutaneous electrode on the person's body by inserting the silver-silver chloride needle electrode subcutaneously into the person's body.

In another aspect of the present invention, a medical tape affixes a cutaneous electrode connector on a person's body. The process of acquiring biopotential signals from a person's body is initiated by coupling at least one connector with a monitoring device where the connector is attached to at least one proximal end of a first wire and a second wire. A cutaneous electrode connector is placed on a surface of the person's body where the cutaneous electrode connector is coupled to a distal end of the second wire and may or may not be connected to a cutaneous electrode. A silver-silver chloride needle electrode is inserted subcutaneously into the person's body if needed where the silver-silver chloride needle electrode is coupled to a distal end of the first wire.

### BRIEF DESCRIPTION OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention, thus the drawings are generalized in form in the interest of clarity and conciseness.

FIG. 1 is a flow diagram showing the method of use of the needle electrode system for acquiring biopotential signals from the person's body.

### DETAILED DESCRIPTION OF THE DRAWINGS

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

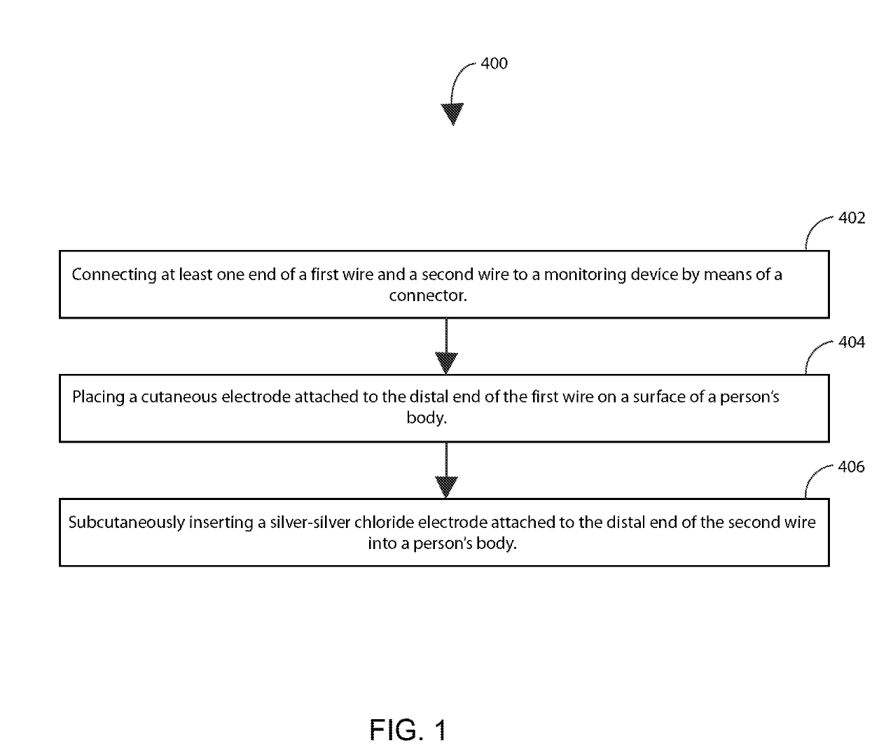

FIG. 1 is a flow diagram 400 showing a method of use of a needle electrode system (not shown) for acquiring biopotential signals (not shown) from a person's body (not shown). In step 402, at least one connector (not shown) is coupled with a monitoring device (not shown) where the connector (not shown) is attached to at least one proximal end (not shown) of a first wire (not shown) and a second wire (not shown). In step 404, a cutaneous electrode connector (not shown) is placed on a surface of the person's body (not shown) where the cutaneous electrode connector (not shown) is coupled to a distal end (not shown) of the second wire (not shown). In step 406, a silver-silver chloride needle electrode (not shown) is inserted subcutaneously into the person's body (not shown) if needed where the silver-silver chloride needle electrode (not shown) is coupled to a distal end (not shown) of the first wire (not shown).

What is claimed is:

1-9. (canceled)

10. A method of using a needle electrode system for acquiring biopotential signals from a person's body, said method comprising:
a) coupling at least one connector with a monitoring device, said connector being attached to at least one proximal end of a first wire and a second wire;
b) placing a cutaneous electrode on a surface of said person's body, said cutaneous electrode connector being coupled to a distal end of said second wire; and
c) inserting a silver-silver chloride needle electrode subcutaneously into said person's body if needed, said silver-silver chloride needle electrode being coupled to a distal end of said first wire.

11. The method of using said needle electrode system of claim 10 wherein said needle electrode system senses and sends biopotential signals from said person's body to said monitoring device.

12. The method of using said needle electrode system of claim 10 wherein a medical tape provides further adhesion to said surface of said person's body.
